(19) United States
(12) Patent Application Publication
(10) Pub. No.: US 2025/0204533 A1
(43) Pub. Date: Jun. 26, 2025

(54) PARTICLE SIZE CONTROLLED CHITOSAN-SILVER ANTIMICROBIAL SOLUTION AND METHOD OF PREPARATION

(71) Applicant: Chitozan Health LLC, Summerville, SC (US)

(72) Inventors: David Juhas, Webster, NY (US); Dwight Austin Van Horn, Summerville, SC (US)

(21) Appl. No.: 18/970,796

(22) Filed: Dec. 5, 2024

Related U.S. Application Data
(60) Provisional application No. 63/613,876, filed on Dec. 22, 2023.

(51) Int. Cl. A01N 59/16; A01N 25/04; A01N 25/10; A61P 1/00

(52) U.S. Cl. CPC A01N 59/16; A01N 25/04; A01N 25/10; A61P 1/00

(57) ABSTRACT

A method for the preparation of an antimicrobial solution comprising the steps of selecting a high molecular weight chitosan; mixing in a reaction vessel said high molecular weight chitosan in acid and water to provide a chitosan solution; placing the chitosan solution in a temperature bath to ensure >90% hydrolyzation; mixing AgNO3 in distilled water to form an AgNO3 solution; mixing NaCl in distilled water to form an NaCl solution; simultaneously subsurface metering the AgNO3 solution and NaCl solution directly to the center of a mixer in the reaction vessel of the chitosan solution following >90% hydrolyzation; mixing the chitosan solution, AgNO3 solution, and NaCl solution in the absence of any white light to form an aggregate solution; cooling the aggregate solution; exposing the aggregate solution to UV light while mixing the aggregate solution; cooling the aggregate solution in a dark refrigerator following UV light exposure.

### GOVERNMENT LICENSE RIGHTS

This invention was made with government support under 1R41AI172693-01 awarded by the National Institutes of Health. The government has certain rights in the invention.

### BACKGROUND OF THE INVENTION

### 1) Field of the Invention

The present invention relates antimicrobial solutions and methods of preparation, and more particularly, to a chitosan-silver based antimicrobial solution having a controlled particle size range and its method of preparation.

### 2) Description of Related Art

Antimicrobial solutions and powders that involve the use of chitosan and silver in certain combinations with other elements are known in the art. For example, U.S. Pat. Nos. 7,344,726 and 7,700,131 disclose methods for preparing chitosan-silver containing solutions. However, a problem with the known methods of the prior art is that the chitosan-silver agglomerations are produced in a wide range of sizes from 4 nm to 900 nm (median 50 nm). The vast majority of the particles produced by these methods are in the nano-particle size range below 100 nm.

There are concerns in the industry with nano-particle chitosan-silver agglomerations below 100 nm as being an environmental pollutant. Accordingly, there is a need for new and novel methods for chitosan-silver antimicrobial solutions that can overcome the problems with particle production having a wide range sized, particularly below 100 nm.

Accordingly, it is an object of the present invention to provide chitosan-silver particle aggregates within a controlled size range above 100 nm for the production of a chitosan-silver based antimicrobial solution.

### SUMMARY OF THE INVENTION

The above objectives are accomplished according to the present invention by providing, in one embodiment, a method for the preparation of an antimicrobial solution comprising the steps of selecting a high molecular weight chitosan; mixing in a reaction vessel said high molecular weight chitosan in acid and water to provide a chitosan solution; placing the chitosan solution in a temperature bath to ensure >90% hydrolyzation; mixing AgNO3 in distilled water to form an AgNO3 solution; mixing NaCl in distilled water to form an NaCl solution; simultaneously subsurface metering the AgNO3 solution and NaCl solution directly to the center of a mixer in the reaction vessel of the chitosan solution following >90% hydrolyzation; mixing the chitosan solution, AgNO3 solution, and NaCl solution in the absence of any white light to form an aggregate solution; cooling the aggregate solution; exposing the aggregate solution to UV light while mixing the aggregate solution; cooling the aggregate solution in a dark refrigerator following UV light exposure.

In a further advantageous embodiment, the method includes selecting the chitosan with a degree of deacetylation greater than 85%.

In a further advantageous embodiment, the method includes selecting the chitosan which has a high molecular weight define by a viscosity greater than 800 mPa in a 1% chitosan solution dissolved in 1% acetic acid.

In a further advantageous embodiment, the method includes selecting the chitosan within the range of between 221-600 kDa.

In a further advantageous embodiment, the method includes selecting the chitosan having about 600 kDa.

In a further advantageous embodiment, the method includes mixing the chitosan with an acid solution of approximately 1% by weight selected from the group consisting of l-lactic, acetic, phosphoric, citric, formic, and malic acids.

In a further advantageous embodiment, the method includes placing the chitosan solution in a 50° C. temperature bath and holding for 480 hours to provide >90% hydrolyzation.

In a further advantageous embodiment, the method includes transferring the chitosan solution to a temperature-controlled reaction vessel where the temperature is maintained between 50° C. and 90° C. and subject to agitation.

In a further advantageous embodiment, the method includes mixing the aggregate solution while maintaining a mixer turnover rate within a range of 18-44 turnovers/min.

In a further advantageous embodiment, the method includes mixing the aggregate solution at a shear rate within a range of 220-390 sec-1.

In a further advantageous embodiment, the method includes running the mixer in the reaction vessel at high mixer speed for approximately 5 minutes then lowering the mixer speed to around 1000 rpm until the aggregate solution is cooled to 25° C.

In a further advantageous embodiment, the method includes exposing the aggregate solution to a 254 nm 55-Watt UV light continuously for about 8 hours.

In a further advantageous embodiment, the method includes precipitating chitosan-silver agglomerations in the aggregate solution predominantly in a size range from 100 nm-350 nm.

In a further advantageous embodiment, the method includes mixing a surfactant of about 0.25% by weight with the aggregate solution.

In a further advantageous embodiment, the method includes mixing with the aggregate solution a cross-linking agent of about 0.375% by weight selected from the group consisting of glutaraldehyde, genipin, citric acid, and uronic acids.

In a further advantageous embodiment, the method includes applying the aggregate solution to a textile for inhibiting antimicrobial resistance.

The objectives are further accomplished according to the present invention by providing, in one embodiment, an antimicrobial solution comprising a chitosan solution having a viscosity greater than 800 mPa in a 1% chitosan solution dissolved in 1% acetic acid; wherein the chitosan solution includes chitosan with a degree of deacetylation greater than 85% and within a range of between 221-600 kDa; and, wherein the chitosan solution has >90% hydrolyzation dissolved in an acid solution of approximately 1% by weight selected from the group consisting of l-lactic, acetic, phosphoric, citric, formic, and malic acids; an aggregate solution including the chitosan solution, an AgNO3 solution and an NaCl solution; and, chitosan-silver particles precipitated in the aggregate solution predominantly in a size range from 100 nm-350 nm.

In a further advantageous embodiment, the aggregate solution was subjected to a mixer turnover rate within a range of 18-44 turnovers/min and with a shear rate within a range of 220-390 sec-1 while in the absence of any white light; and, wherein the aggregate solution was subjected to UV light exposure.

In a further advantageous embodiment, the antimicrobial solution includes a surfactant of about 0.25% by weight, and a cross-linking agent of about 0.375% by weight selected from the group consisting of glutaraldehyde, genipin, citric acid, and uronic acids.

In a further advantageous embodiment, the antimicrobial solution is disposed on a textile.

### BRIEF DESCRIPTION OF THE DRAWINGS

The system designed to carry out the invention will hereinafter be described, together with other features thereof. The invention will be more readily understood from a reading of the following specification and by reference to the accompanying drawings forming a part thereof, wherein an example of the invention is shown and wherein:

FIG. 1 shows TEM image of silver chloride particle aggregates surrounded by chitosan, wherein the dark shapes are silver chloride particles held together by organic material and silver-silver dipole forces according to the present invention.

It will be understood by those skilled in the art that one or more aspects of this invention can meet certain objectives, while one or more other aspects can meet certain other objectives. Each objective may not apply equally, in all its respects, to every aspect of this invention. As such, the preceding objects can be viewed in the alternative with respect to any one aspect of this invention. These and other objects and features of the invention will become more fully apparent when the following detailed description is read in conjunction with the accompanying figures and examples. However, it is to be understood that both the foregoing summary of the invention and the following detailed description are of a preferred embodiment and not restrictive of the invention or other alternate embodiments of the invention. In particular, while the invention is described herein with reference to a number of specific embodiments, it will be appreciated that the description is illustrative of the invention and is not constructed as limiting of the invention. Various modifications and applications may occur to those who are skilled in the art, without departing from the spirit and the scope of the invention, as described by the appended claims. Likewise, other objects, features, benefits and advantages of the present invention will be apparent from this summary and certain embodiments described below, and will be readily apparent to those skilled in the art. Such objects, features, benefits and advantages will be apparent from the above in conjunction with the accompanying examples, data, figures and all reasonable inferences to be drawn therefrom, alone or with consideration of the references incorporated herein.

### DETAILED DESCRIPTION OF A PREFERRED EMBODIMENT

With reference to the drawings, the invention will now be described in more detail. Unless defined otherwise, all technical and scientific terms used herein have the same meaning as commonly understood to one of ordinary skill in the art to which the presently disclosed subject matter belongs. Although any methods, devices, and materials similar or equivalent to those described herein can be used in the practice or testing of the presently disclosed subject matter, representative methods, devices, and materials are herein described.

Unless specifically stated, terms and phrases used in this document, and variations thereof, unless otherwise expressly stated, should be construed as open ended as opposed to limiting. Likewise, a group of items linked with the conjunction “and” should not be read as requiring that each and every one of those items be present in the grouping, but rather should be read as “and/or” unless expressly stated otherwise. Similarly, a group of items linked with the conjunction “or” should not be read as requiring mutual exclusivity among that group, but rather should also be read as “and/or” unless expressly stated otherwise.

Furthermore, although items, elements or components of the disclosure may be described or claimed in the singular, the plural is contemplated to be within the scope thereof unless limitation to the singular is explicitly stated. The presence of broadening words and phrases such as “one or more,” “at least,” “but not limited to” or other like phrases in some instances shall not be read to mean that the narrower case is intended or required in instances where such broadening phrases may be absent.

The new solution and method for preparation of the present invention is a novel technique where silver chloride crystal aggregates tightly adhere to each other by dipole-dipole attraction with the chitosan polymer bonding itself to the aggregate by strong Van der Waals forces. This unique feature differentiates the present invention from the prior art by limiting the formation of “free silver” observed in alternative silver formulations used in antimicrobial applications. Ultimately, the present invention provides the ability to control the size range of the silver particle aggregates for the production of a chitosan-silver antimicrobial solution.

The particle size controlled chitosan-silver solution and the method for its production according to the present invention is a highly differentiated solution produced using manufacturing methodology new to the art. The present invention provides a method for preparation that continues to bind silver onto the free amine groups of the chitosan, the resultant chitosan-silver is no longer produced in an unpredictable range of elemental silver and chitosan-silver agglomerations from 4 nm to 900 nm (median 50 nm). Instead, the new chitosan-silver is produced with a controlled and predictable range from approximately 150 nm to 350 nm (median 230 nm) of dipole-dipole attached silver chloride aggregates. The median size can be controlled from approximately 50 to 350 nm by varying key process variables. This new formulation and process produces particles within a controlled precipitation reaction, forming aggregates well above the nano-silver range. Most prior art produces nano silver chitosan via chemical or light reduction of silver nitrate in the presence of chitosan amine groups. Those methods predominantly produce particles well below 100 nm. This new process inhibits reduction to enable silver chloride precipitates with sizes well above 100 nm, typically between approximately 150 nm to 350 nm in accordance with example embodiments described herein. The new chitosan-silver particles of the present invention support the creation of chitosan-silver aggregate sizes that can be used to avoid the issues of uncontrolled particle sizes. As the present invention establishes median aggregate sizes above approximately 230 nm, the probability of seeing particles below 100 nm is extremely small with the methods of preparation detailed herein.

The features of an antimicrobial solution and method for its preparation according to the present invention include 1) using high molecular weight chitosan only (no low or medium); 2) higher chitosan concentrations with increased hydrolysis produces larger aggregates; 3) simultaneous additions of dilute silver nitrate and sodium chloride solutions; 4) particles formed via controlled precipitation reaction and not reduction; 5) median size can be controlled from approximately 50 to 350 nm by varying process variables; 6) simultaneously mixing the chitosan and reactants; 7) mixing the chitosan and silver at specific rates; 8) mixing by a subsurface means; subsurface mixing at specific vessel turnover rates; 9) subsurface addition of reactant solutions directly into a high shear mixer; 10) mixing at elevated temperatures 60° C. and greater; 11) mixing in non-white light conditions; 12) UV exposures from 8-12 hours driving increased silver-silver dipole attractions wherein the silver is fully attached to chitosan amine groups; chitosan-silver chloride agglomerations greater than 100 nm; 13) minimal amount of silver usage yet highly antimicrobial; and, 14) overall less use of chitosan and silver amounts without loss of antimicrobial effectiveness.

Using a transmission electron microscopy (TEM), a comparison is shown in Table 1 of particles produced by the process known in the art (see for example U.S. Pat. Nos. 7,344,726 and 7,700,13) versus particles produced according to the process of the present invention described herein. The old process was highly variable, unpredictable, and was not readily controlled. This is established by TEM imaging performed by the GA Tech, Materials Characterization Facility in Atlanta, GA.

TABLE 1
TEM percentage of nanoparticles greater than 100
nm, 200 nm, and 300 nm-Old Art vs New Process

| | | New Process |
|---|---|---|
| | Prior Art Median = 60 nm | Median = 230 nm |
| Size Range | N = 400 | N = 138 |
| >100 nm | 23% | 100% |
| >200 nm | 6% | 73% |
| >300 nm | 2% | 17% |

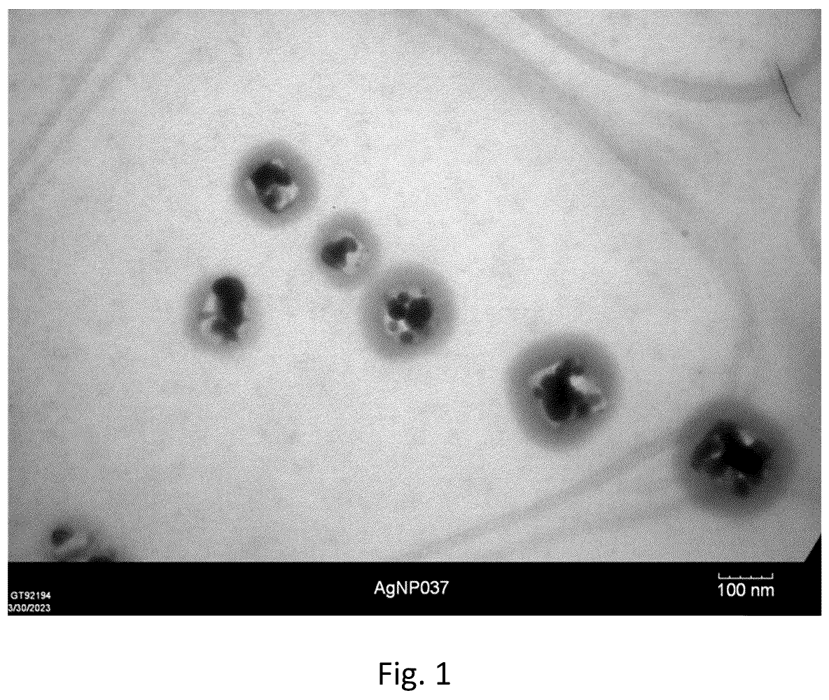

Referring to FIG. 1, A TEM image is shown to illustrate the chitosan-silver attachment of the present invention.

Chitosan is a deacetylated derivative of chitin. Chitin is an aminated polysaccharide biosynthesized in several invertebrate animal species. It is a main compound of the exoskeleton of arthropods, the most abundant animal phyla that include insects and crustaceans. Chitosan is the term used to denominate the polymer of glucosamine and N-acetyl glucosamine where the deacetylated units are present in major proportion or their distribution in the polymer chain is such that allows it to be dissolved in aqueous diluted acid solutions. A distinctive feature of the chemical structure of chitosan is the predominant presence of units with amino groups that can be ionized. These groups become cationic in acidic media promoting the chitosan dissolution and polyelectrolyte behavior in solution. Chitosan is generally characterized by the Degree of deacetylation (DDA) and it's Molecular Weight (MW), where both the chitin source and method of deacetylation can also alter the final properties.

The greater the DDA, the more amine groups are formed, the greater adhesion to both the particle and the textile it will be applied to. The present invention is focused on DDA's greater than 85%, and up to about 95%. Chitosan MW is generally characterized as the viscosity of a 1% chitosan solution dissolved in 1% acetic acid. The actual molecular weight can be calculated through analytical means and correlates directly to viscosity. High molecular weight (HMW) is generally categorized in a viscosity range of 800-2400 mPa or a molecular weight of approximately 600 kDa, Medium Molecular Weight (MMW) as 200-800 mPa or ˜220 kDa, and Low Molecular Weight (LMW) as 20-200 mPa˜15 kDa. These numbers vary with manufacturer and within manufacturing. The present invention explored MW from 70 to 1600 kDa, but found the best range between 160-600 kDa, with 600 kDa (HMW) being preferred. LMW was avoided due to excessive levels of melanoidins and their propensity to broaden particle size distributions. Melaniodins are formed when chitosan is exposed to elevated temperatures which is generally required to lower its molecular weight. In one embodiment, it was also desirable to establish a final viscosity >1000 mPa for product stability of the larger particle sizes.

The High Molecular Weight chitosan is dissolved in a dilute weak acid solution of approximately 1% by weight l-lactic acid. Acetic, phosphoric, citric, formic, and malic acids may also be used, however l-lactic acid provides excellent hydrolyzation properties with no stringent odors and is thus preferred. It's also widely used in the cosmetic industry, an EPA safer use chemical, and readily available at reasonable costs.

In one arrangement, aqueous solutions of approximately 1-1.5% by weight high molecular weight chitosan are created resulting in a relatively high viscosities (1000-2400 mPa respectively). In the presence of a weak acid, the amine groups in chitosan will hydrolyze enabling the chitosan to go into solution. A unique characteristic in chitosan is that it will become a solution when only 50% hydrolyzed. However, in the presence of l-lactic acid will continue to hydrolyze, reducing the molecular weight and viscosity over time. Controlling the degree of hydrolyzation of the chitosan solution impacts particle size, distribution, and viscosity. Hydrolyzation is managed by heating the chitosan lactic acid solution at 50° C. for a time period resulting in 40-97% viscosity loss. Viscosity loss decreases logarithmically with time and more rapidly at higher temperatures, however exceeding 53° C. results in melanoidin formation. Hydrolyzation can occur at room temperature but for longer times. The resulting 100-600 mPa mixture can be filtered down to 1.5 microns. Viscometry is used to characterize molecular weight change through hydrolysis. Increasing chitosan concentration increases aggregate size and increases the chitosan shell surrounding the aggregate (See FIG. 1). Increasing hydrolysis enables lower viscosity solutions of higher concentrations improving reactor vessel turnover rate. It also reduces post precipitation viscosity loss, with rates decreasing logarithmically with degree of hydrolyzation, thus improving product stability.

To robustly precipitate chitosan silver agglomerations with particle sizes greater than 100 nm, process control of reactant flows, temperature and mixing are essential. In an example embodiment, silver nitrate at approximately 0.062% by weight and sodium chloride of 0.021% by weight solutions are slowly metered at equal molar flowrates subsurface directly to the mix point (0.7 millimole/min over 6.5 minutes for 1.25 L batch) with the chitosan solution. A high shear, non-foaming mixer is used to produce high radial flows, enabling various turnover rates (in one embodiment, ˜77 turnovers/min, ranging from about 60-106) and the highest shear precisely (˜280 sec⁻¹) where the reactants come together (mix point). In a preferred embodiment, the measured turnover rate is within a range of 18-44 turnovers/min, and preferably approximately 30 turnovers/min. The mixer head is preferably designed to handle higher viscosities associated with high molecular weight chitosan enabling viscosities as high as 600 cp. Slow reactant addition at increased solubility restrains nucleation thus preventing smaller crystal formation and enabling the silver chloride to bind directly to the more stable non-hydrolyzed amine groups along the chitosan backbone. High solubility is established by increasing both chitosan concentration and the reaction temperature (60-90° C.). Controlling these factors, in addition to the degree of hydrolyzation, enables larger silver chloride aggregate medians (chitosan silver agglomerations particularly in the 100-350 nm size range) to form without the presence of smaller silver chloride crystals. Further, at no time during the processing is the solution exposed to white-light conditions to aid in silver size control.

This process is also capable of achieving controlled median particle sizes for the chitosan silver agglomerations below 100 nm by varying temperature and reactant flow rates. However, the invention goal was to create median sizes above 100 nm.

The final criteria for creating particle sizes with the 100-350 nm range is exposure to 55-Watt 254 nm UV light for a controlled length of time (8-12 hours). The UV irradiation initiates AgCl reduction to Ag at the particle surface. The total shell Ag concentration increasing to 3-7% at 8 to 12 hours respectively. This silver outer layer, not only enhances the chelation attractions with the chitosan, but more importantly creates somewhat stronger dipole attractions between aggregated particles. As observed in the TEM image, the end-to-end dipole aggregates undergo Oswald ripening melding the particles within the aggregate together.

The key process variables and their ranges are listed below in Table 2.

TABLE 2
Key Process Variables with their Preferred Condition and Working Ranges

| Process Variable | Preferred | Range |
|---|---|---|
| % Viscosity Hydrolyzation | >90% | 40-97% |
| Precipitation Temperature (° C.) | 70 | 50-90 |
| Mixer Turnovers/min | 30 | 18-44 |
| Mixer Shear Rate (sec⁻¹) | 280 | 220-390 |
| Reactant Molar Flow (mMoles/m) | 0.7 | 0.07-3.2 |
| Total Ag Moles Added (mMoles) | 6.09 | 4.4-50 |
| Excess NaCl (g/L) | 0.003 | .002-4.2 |
| pH | 3.4 | 2.5-5.3 |
| UV Irradiation (hrs) | 8 | 0-15 |

This solution can then be made into various forms including serums, gels and foams or dried into a powder or coated onto various fabrics. Coating on textiles requires a small amount of Dow Chemical NP-9 Surfactant (˜0.25% by weight) or equivalent to aid solution spreading onto fabrics. A cross-linking agent is necessary to minimize chitosan dissolution during washing. Glutaraldehyde (˜0.375% by weight) is used although alternatives are acceptable such as Genipin, citric acid, and uronic acids.

The current coating process (Padding Method) requires full fabric saturation with excess material removed via pressure rollers. The fabric is currently left to air dry but could be dried at elevated temperatures up to 115° C. Different materials will absorb the product differently requiring silver laydown management per fabric type and weight.

The new process and solution of the present invention are only capable by the discovery and control of new and key processing variables that were not previously understood or described in earlier chitosan-silver manufacturing and intellectual property of the prior art. The present invention ensures all silver is attached to the chitosan amine groups preventing particle release readily seen in alternative commercial products that are not attached. The amount of coated silver loadings used (0.015-0.055% by weight) are low compared to other approved antimicrobial silver products commercially available and when used in combination with chitosan results in impressive antimicrobial properties providing a greater than 99.9% effective kill rate against a variety of deadly microorganisms as shown below in Table 3.

TABLE 3
Chitosan-silver treated fabrics and log reductions against various microorganisms

| Material | Test Method | Pathogen | Strain | % reduction | log reduction | time point |
|---|---|---|---|---|---|---|
| 100% cotton | ASTM E2149-13a | E coli | ATCC 25922 | 99.95 | >3 log 10 | 2 hour |
| 100% cotton | ASTM E2149-13a | K pneumoniae | ATCC 4352 | 99.98 | >3 log 10 | 2 hour |
| 100% cotton | ASTM E2149-13a | MRSA | ATCC 33591 MR | 99.97 | >3 log 10 | 2 hour |
| 100% cotton | ASTM 82149-13a | Staph aureus | ATCC 6538 | 99.95 | >3 log 10 | 2 hour |
| Rigid Poly Twist | ASTM E2149-13a | E coli | ATCC 17218 | 99.95 | >3 log 10 | 2 hour |
| Rigid Poly Twist | ASTM E2149-13a | K pneumoniae | ATCC 38788 | 99.98 | >3 log 10 | 2 hour |
| Rigid Poly Twist | ASTM E2149-13a | MRSA | ATCC 33591 MR | 99.97 | >3 log 10 | 2 hour |
| Rigid Poly Twist | ASTM E2149-13a | Staph aureus | ATCC 6539 | 99.98 | >3 log 10 | 2 hour |
| Nylon Zwick stretch | ASTM E2149-13a | E coli | ATCC 25922 | 99.95 | >3 log 10 | 2 hour |
| Nylon Zwick stretch | ASTM E2149-13a | K pneumoniae | ATCC 4352 | 99.98 | >3 log 10 | 2 hour |
| Nylon Zwick stretch | ASTM E2149-13a | MRSA | ATCC 33591 MR | 99.97 | >3 log 10 | 2 hour |
| Nylon Zwick stretch | ASTM 82149-13a | Staph aureus | ATCC 6538 | 99.98 | >3 log 10 | 2 hour |
| Nylon Spandex | ASTM E2149-13a | E coli | ATCC 17218 | 99.95 | >3 log 10 | 2 hour |
| Nylon Spandex | ASTM E2149-13a | K pneumoniae | ATCC 38788 | 99.98 | >3 log 10 | 2 hour |
| Nylon Spandex | ASTM E2149-13a | MRSA | ATCC 33591 MR | 99.95 | >3 log 10 | 2 hour |
| Nylon Spandex | ASTM E2149-13a | Staph aureus | ATCC 6539 | 99.95 | >3 log 10 | 2 hour |

Note that the coated silver coverage depends on the fabric type and associated fabric weight (GSM). Our silver coverage varies from 20 to 60 mg/m² on multiple fabrics tested (nylon-spandex, cotton, polyester, and nylon).

The chitosan concentration can vary, however higher concentrations with longer hydrolysis times are preferred as shown below in Table 4.

TABLE 4
New Chitosan-silver chemical ingredients and unit measures for
preferred 6.09 milli-Mole Precipitation

| | | | | Preferred | Preferred |
|---|---|---|---|---|---|
| | preferred | Preferred Amount | Working Range | Final | Final |
| Ingredient | CAS# | (grams) | (grams) | (grams) | (g/Kg) |
| Silver Nitrate | 7761-88-8 | 1.035 | 0.74-8.5 | 0 | 0 |
| Sodium Chloride | 7647-14-5 | 0.359 | 0.26-4.15 | 0.00359 | 0.003 |
| Chitosan (HMw) | 9012-76-4 | 12.94 | 6-12.94 | 12.94 | 9.771 |
| L-Lactic Acid | 79-33-4 | 14.7 | 10-22 | 14.7 | 11.1 |
| Potassium Sorbate | 24634-61-5 | 1.3 | 0.8-1.7 | 1.3 | 0.982 |
| Distilled Water | 7732-18-5 | 1294 | 800-1700 | 1294 | 977.1 |
| Silver Chloride | 7783-90-6 | | | 0.873 | 0.659 |
| Bonded Silver | 7440-22-4 | | | 0.026 | 0.02 |
| Sodium Nitrate | 7631-99-4 | | | 0.518 | 0.391 |

The new chitosan-silver solution and method for production described herein results in size controlled chitosan silver agglomeration particles. The process described herein can be scaled, and the end-product is one that can deliver chitosan-silver agglomerations predominantly with sizes greater than 100 nm, particularly in the 100 nm-350 nm size range. While chitosan-silver solutions are previously described, the process results in random attachment and chitosan silver agglomeration particle sizes predominately as a nano-material size below 100 nm not beneficial to the environment and without complete silver to chitosan attachment.

The present invention is further illustrated by the following example embodiment for providing chitosan-silver agglomerations predominantly with sizes greater than 100 nm, particularly in the 100 nm-350 nm size range.

### Example 1

12.94 gram of HMW chitosan is slowly added to a mixture of 14.7 grams of l-lactic acid in 872.4 grams of distilled water. The solution is mixed for 8 hours, then places in a 50° C. temperature bath and held for 480 hours to ensure >90% hydrolyzation. The mixture is transferred to a 2 L temperature-controlled reaction vessel where the temperature is increased to 70° C. under low agitation. Meanwhile an AgNO3 solution is prepared by adding 1.035 grams dry AgNO3 to 167 grams distilled water and mixed in a 70° C. beaker. A NaCl solution is prepared by adding 0.359 grams of dry NaCl to 166.8 grams of distilled water and placed and mixed in a separate 70° C. beaker. Once the reaction vessel has reached 70° C., the lights are turned off (red lights only), and the mixer brought to speed measuring 30 turnovers/min. The salt and silver solutions are connected to peristaltic pumps which in turn meter the solutions directly to the center of the mixer (shear rate of 280 sec⁻¹). The salt solution starts first and is controlled to 19.09 cc/min (0.7 millimoles/min). After 5 seconds, the AgNO3 solution begins flowing also at 19.09 cc/min (0.7 millimoles/min). The reaction continues for 8.7 minutes. Once the reactant beakers are run dry, and additional quantity of 30 cc's distilled water is chased through each reactant line driving all of the reactants into the reaction vessel. During the reactant addition, the mixer speed gradually increases to maintain a constant vessel turnover rate. Once the lines have been purged the pumps automatically turn off and the reaction is complete. The vessel is run at high mixer speed for 5 minutes then it is lowered to around 1000 rpm and the cooling process to 25° C. is begun (approximately 15-20 minutes). The solution is then transferred to a chamber for UV light exposure. Within this chamber the solution continues to be mixed at low speed. The 254 nm 55-Watt light is turned on and held on for 8 hours. Once the 8-hour irradiation is complete, the solution is weighed, measured for viscosity and pH, then placed in a dark refrigerator.

While the present subject matter has been described in detail with respect to specific exemplary embodiments and methods thereof, it will be appreciated that those skilled in the art, upon attaining an understanding of the foregoing may readily produce alterations to, variations of, and equivalents to such embodiments. Accordingly, the scope of the present disclosure is by way of example rather than by way of limitation, and the subject disclosure does not preclude inclusion of such modifications, variations and/or additions to the present subject matter as would be readily apparent to one of ordinary skill in the art using the teachings disclosed herein. The scope of the invention should, therefore, be determined not with reference to the above description, but should instead be determined with reference to the appended claims, along with the full scope of equivalents to which such claims are entitled. The disclosures of all articles and references, including patent applications and publications, are incorporated by reference for all purposes. The omission in the following claims of any aspect of subject matter that is disclosed herein is not a disclaimer of such subject matter, nor should it be regarded that the inventor did not consider such subject matter to be part of the disclosed inventive subject matter.

What is claimed is:

1. A method for the preparation of an antimicrobial solution, the method comprising the steps of:
selecting a high molecular weight chitosan;
mixing in a reaction vessel said high molecular weight chitosan in acid and distilled water to provide a chitosan solution;
placing the chitosan solution in a temperature bath to reach >90% hydrolyzation;
mixing AgNO3 in distilled water to form an AgNO3 solution;
mixing NaCl in distilled water to form an NaCl solution;
simultaneously subsurface metering the AgNO3 solution and NaCl solution directly to the center of a mixer in the reaction vessel of the chitosan solution following >90% hydrolyzation;
mixing the chitosan solution, AgNO3 solution, and NaCl solution in the absence of any white light to form an aggregate solution;
cooling the aggregate solution;
exposing the aggregate solution to UV light while mixing the aggregate solution;
cooling the aggregate solution in a dark refrigerator following UV light exposure.

2. The method of claim 1 including selecting the chitosan with a degree of deacetylation greater than 85%.

3. The method of claim 1 including selecting the chitosan which has a high molecular weight define by a viscosity greater than 800 mPa in a 1% chitosan solution dissolved in 1% acetic acid.

4. The method of claim 1 including selecting the chitosan within the range of between 221-600 kDa.

5. The method of claim 1 including selecting the chitosan having about 600 kDa.

6. The method of claim 1 including mixing the chitosan with an acid solution of approximately 1% by weight selected from the group consisting of l-lactic, acetic, phosphoric, citric, formic, and malic acids.

7. The method of claim 1 including placing the chitosan solution in a 50° C. temperature bath and holding for 480 hours to provide >90% hydrolyzation.

8. The method of claim 1 including transferring the chitosan solution to a temperature-controlled reaction vessel where the temperature is maintained between 50° C. and 90° C. and subject to agitation.

9. The method of claim 1 including mixing the aggregate solution while maintaining a mixer turnover rate within a range of 18-44 turnovers/min.

10. The method of claim 1 including mixing the aggregate solution at a shear rate within a range of 220-390 sec₋₁.

11. The method of claim 1 including running the mixer in the reaction vessel at high mixer speed for approximately 5 minutes then lowering the mixer speed to around 1000 rpm until the aggregate solution is cooled to 25° C.

12. The method of claim 1 including exposing the aggregate solution to a 254 nm 55-Watt UV light continuously for about 8 hours.

13. The method of claim 1 including precipitating chitosan-silver agglomerations in the aggregate solution predominantly in a size range from 100 nm-350 nm.

14. The method of claim 13 including mixing a surfactant of about 0.25% by weight with the aggregate solution.

15. The method of claim 14 including mixing with the aggregate solution a cross-linking agent of about 0.375% by weight selected from the group consisting of glutaraldehyde, genipin, citric acid, and uronic acids.

16. The method of claim 15 including applying the aggregate solution to a textile for inhibiting antimicrobial resistance.

17. A method for the preparation of an antimicrobial solution, the method comprising the steps of:
selecting a high molecular weight chitosan;
mixing the chitosan in acid and distilled water to provide a chitosan solution;
mixing AgNO3 in distilled water to form an AgNO3 solution;
mixing NaCl in distilled water to form an NaCl solution;
mixing the chitosan solution, AgNO3 solution, and NaCl solution together in the absence of any white light to form an aggregate solution;
exposing the aggregate solution to UV light while mixing the aggregate solution; and,
cooling the aggregate solution in a dark refrigerator following UV light exposure.

18. The method of claim 17 including selecting the chitosan with a degree of deacetylation greater than 85% and having about 600 kDa.

19. The method of claim 17 including mixing the chitosan with an l-lactic acid solution of approximately 1% by weight and the distilled water.

20. The method of claim 17 including placing the chitosan solution in a 50° C. temperature bath and holding for 480 hours to provide >90% hydrolyzation.

21. The method of claim 17 including simultaneously subsurface metering the AgNO3 solution and NaCl solution directly to the center of a mixer in a reaction vessel following >90% hydrolyzation of the chitosan solution.

22. The method of claim 17 including mixing the aggregate solution while maintaining a mixer turnover rate within a range of 18-44 turnovers/min and a shear rate within a range of 220-390 sec⁻¹.

23. The method of claim 17 including cooling the aggregate solution to about 25° C. and then exposing the aggregate solution to a 254 nm 55-Watt UV light continuously for about at least 8 hours.

24. The method of claim 17 including precipitating chitosan-silver agglomerations in the aggregate solution predominantly in a size range from 100 nm-350 nm.

25. An antimicrobial solution comprising:
a chitosan solution having a viscosity greater than 800 mPa in a 1% chitosan solution dissolved in 1% acetic acid;
wherein the chitosan solution includes chitosan with a degree of deacetylation greater than 85% and within a range of between 221-600 kDa; and,
wherein the chitosan solution has >90% hydrolyzation dissolved in an acid solution of approximately 1% by weight selected from the group consisting of l-lactic, acetic, phosphoric, citric, formic, and malic acids;
an aggregate solution including the chitosan solution, an AgNO3 solution and an NaCl solution; and,
chitosan-silver particles precipitated in the aggregate solution predominantly in a size range from 100 nm-350 nm.

26. The antimicrobial solution of claim 25 wherein the aggregate solution was subjected to a mixer turnover rate within a range of 18-44 turnovers/min and with a shear rate within a range of 220-390 sec⁻¹ while in the absence of any white light; and, wherein the aggregate solution was subjected to UV light exposure.

27. The antimicrobial solution of claim 25 including a surfactant of about 0.25% by weight, and a cross-linking agent of about 0.375% by weight selected from the group consisting of glutaraldehyde, genipin, citric acid, and uronic acids.

28. The antimicrobial solution of claim 27 wherein the antimicrobial solution is disposed on a textile.
